(19) United States
(12) Patent Application Publication
(10) Pub. No.: US 2010/0076320 A1
(43) Pub. Date: Mar. 25, 2010

(54) FLUSH CATHETER WITH FLOW DIRECTING SHEATH

(75) Inventors: Christopher L. Petersen, Carlisle, MA (US); Michael Atlas, Arlington, MA (US); Christopher Petroff, Groton, MA (US)

Correspondence Address: K&L Gates LLP, STATE STREET FINANCIAL CENTER, One Lincoln Street, BOSTON, MA, 02111-2950, US

(73) Assignee: LIGHTLAB IMAGING, LLC, Westford, MA (US)

(21) Appl. No.: 12/565,332

(22) Filed: Sep. 23, 2009

Related U.S. Application Data
(63) Continuation-in-part of application No. 11/822,600, filed on Jul. 9, 2007, now Pat. No. 7,625,366.
Application No. 11/822,600 is a continuation of application No. 10/423,016, filed on Apr. 25, 2003, now Pat. No. 7,241,286.

(51) Int. Cl. A61M 25/00; A61B 6/00

(52) U.S. Cl. 600/478; 604/508

(57) ABSTRACT

In certain embodiments, the invention provides a method of flushing a lumen of interest having a first diameter and a lumen wall. The method can include the steps of selecting a flush solution such that the flush solution lowers a fluid removal rate of a plurality of terminating lumens, the terminating lumens branching from and in fluid communication with the lumen of interest, at least one of the terminating lumens having a second diameter, the second diameter smaller than the first diameter; flushing the lumen with the flush solution; and collecting optical tomography scan data relative to a portion of the lumen wall.

### REFERENCE TO RELATED APPLICATIONS

This application is a continuation-in-part of U.S. application Ser. No. 11/822,600, filed Jul. 9, 2007, which is a continuation of U.S. application Ser. No. 10/423,016, filed Apr. 25, 2003, now U.S. Pat. No. 7,241,286, the entire contents of each of which are hereby incorporated by reference herein.

### FIELD OF THE INVENTION

The invention is directed to a flush catheter and, more particularly, to a flush catheter provided with a flow directing sheath.

### BACKGROUND OF THE INVENTION

In order to obtain clear in-vivo images of arterial walls when using, for example, Optical Coherence Tomography (OCT), it is necessary to displace blood from a cylindrical volume around a tip of an imaging probe. To allow surveying of a length of an artery wall, it is desirable that the cylindrical volume be, for example, as long as approximately 40-50 mm or more. The better the blood is cleared from this volume, the better the image obtained of the arterial wall.

For example, in almost all uses of OCT for imaging during cardiac catherizations, an imaging probe disposed within a guide catheter is inserted into an artery such that a direction of blood flow is from a proximal end of the imaging probe toward a distal end of the catheter or probe. It is desirable that a location of the cleared cylindrical volume be somewhat proximal to the distal end of the catheter, to allow the use of a “minirail” delivery system. A “minirail” delivery system utilizes a guide wire and a flexible tip attachable to the imaging probe. The guide wire is used to guide the imaging probe into the desired artery.

Previous and current methods of achieving the desired cleared volume or blood displacement have included the use of cardiac dilation balloons, the injection of saline through a guide catheter, and the injection of saline through a selective flush catheter inserted over the imaging catheter. All three of these methods provide less than ideal solutions.

The balloon method either involves total occlusion of a vessel for the time that the image is desired, or the use of under-inflated balloons which does not completely remove the blood from the field of view. The guide flush method requires a large flow rate of saline that can over hydrate the patient. This method is also very ineffective when side branches are present.

For example, when blood flow is from a proximal to a distal end of the imaging probe, the selective flush catheter method has the inherent limitation that blood from the area proximal to the flush point is entrained into the flush solution at a point where the flush solution exits the catheter. Increasing the flow rate of flush solution tends to entrain more blood, making it difficult to dilute the blood enough to provide a clear imaging area. In addition, it is difficult to configure this type of device for a minirail delivery system.

U.S. Pat. No. 4,878,893 (hereinafter “the 893 patent”) to Albert K. Chin entitled “Angioscope with Flush Solution Deflector Shield,” which is hereby incorporated by reference, provides a partial solution to this problem, and is intended for use with an angioscope catheter. The 893 patent teaches the use of a curved deflector shield 30 bonded to a distal tip of a catheter 10. The deflector shield 30: . . . causes the flushing solution to momentarily flow against blood flow toward the proximal end of the catheter. The blood flow will then carry the solution back past the distal tip of the angioscope 18, as shown in FIG. 13 [of the 893 patent] to provide the bolus required for clear visualization as discussed at col. 5, lines 1-6, of the 893 patent. However, the approach of the '893 patent has several deficiencies which prevent its use in an OCT application and which make it difficult to produce.

For example, the deflector shield must be at a distal end of the catheter, making it difficult to use a minitail type of delivery system. Further, the design does not strongly direct the flushing solution in an axial proximal direction. This results in much of the flushing solution moving out from the catheter in a radial direction. As such, the bolus of flushing solution does not flow very far toward the proximal end of the catheter and will not provide the long volume desirable for surveying a length of the artery wall. Furthermore, radially directed jets of fluid can damage the sensitive endothelial layer of the vessel and could even perforate the vessel.

The above references are incorporated by reference herein where appropriate for appropriate teachings of additional or alternative details, features and/or technical background.

### SUMMARY OF THE INVENTION

An object of the invention is to solve at least the above problems and/or disadvantages and to provide at least the advantages described hereinafter.

The invention is directed to a flush catheter, and more particularly, to a flush catheter with a flow directing sheath.

In certain embodiments, the invention provides a flush catheter configured to be introduced into a lumen to create an optically transparent flush zone. The flush catheter can include a catheter body configured to be introduced into a lumen and an inner cavity, the inner cavity being configured to communicate with a proximal source of flush solution and expel the flush solution at a distal end of the catheter; an image probe assembly contained within the catheter body; and a plurality of openings provided in the catheter body configured to expel therethrough the flush solution; wherein when flush solution is expelled through the plurality of openings into the lumen, the flush solution is directed to flow toward a proximal end of the catheter body, and wherein a flush solution flow rate is configured such that a volume flow rate of the expelled flush solution is substantially equivalent but opposite to that of locally flowing blood thereby creating the optically transparent flush zone along a length of the lumen such that non-occlusive optical imaging of the lumen can be performed by the image probe assembly.

In some embodiments of the flush catheter, the flush solution comprises iodine having a concentration that ranges from about 150 mg/ml to 400 mg/ml.

In some embodiments of the flush catheter, the flush solution has a viscosity that ranges from about 3 cps to about 9 cps at body temperature.

In some embodiments of the flush catheter, a viscosity of the flush solution does not vary substantially with temperature.

In some embodiments of the flush catheter, the flush solution comprises dextran.

In some embodiments of the flush catheter, the flush solution comprises a dextran concentration that ranges from 5% to 20%, with a molecular weight of 20,000 to 100,000 Daltons.

In certain embodiments, the invention provides a method of flushing a lumen of interest having a first diameter and a lumen wall. The method can include the steps of selecting a flush solution such that the flush solution lowers a fluid removal rate of a plurality of terminating lumens, the terminating lumens branching from and in fluid communication with the lumen of interest, at least one of the terminating lumens having a second diameter, the second diameter smaller than the first diameter; flushing the lumen with the flush solution; and collecting optical tomography scan data relative to a portion of the lumen wall.

In some embodiments of the method, the flush solution has been further selected to remove blood near the portion of the lumen wall.

In some embodiments of the method, the flush solution comprises dextran.

In some embodiments of the method, the terminating lumens are capillaries.

In some embodiments of the method, the flush solution has a viscosity that ranges from about 3 cps to about 9 cps at body temperature.

In some embodiments of the method, the flush solution is a radio-opaque contrast solution.

In some embodiments of the method, an iodine concentration of the contrast solution ranges from about 150 mg/ml to 400 mg/ml.

In some embodiments of the method, the contrast solution comprises iodine having a concentration from about 150 mg/ml to about 400 mg/ml.

Additional advantages, objects, and features of the invention will be set forth in part in the description which follows and in part will become apparent to those having ordinary skill in the art upon examination of the following or may be learned from practice of the invention. The objects and advantages of the invention may be realized and attained as particularly pointed out in the appended claims.

### BRIEF DESCRIPTION OF THE DRAWINGS

The invention will be described in detail with reference to the following drawings in which like reference numerals refer to like elements wherein:

FIG. 1 is a schematic, partial, side, perspective view of a flush catheter implemented in combination with an imaging probe according to an embodiment of the invention;

FIG. 2 is a schematic, partial, side, cross-sectional view of the flush catheter implemented in combination with an imaging probe of FIG. 1;

FIG. 3 is another schematic, partial, side, perspective view of the flush catheter implemented in combination with an imaging probe of FIG. 1;

FIG. 4 is another schematic, partial, side, cross-sectional perspective view of the flush catheter implemented in combination with an imaging probe of FIG. 1;

FIG. 5 is a schematic, partial, side view of the flush catheter implemented in combination with an imaging probe of FIG. 1;

FIG. 6 is a schematic, partial, side, cross-sectional view of the flush catheter implemented in combination with an imaging probe of FIG. 1; and

FIG. 7 is an enlarged, schematic, side, cross-sectional view of the sheath according to the invention.

### DETAILED DESCRIPTION

The invention is directed to a flush catheter configured to be inserted into an artery, vessel, or other orifice in a patient. In addition, embodiments of the invention are also directed to the use of flush solutions having predetermined viscosities.

Blood has a viscosity of approximately 3-4 centipoise (cps), dependant primarily on the hematocrit level. In contrast, water, by definition, has a viscosity of 1.00 cps. One of the primary contributors to flow resistance and hence blood pressure, is the resistance of the fine-diameter capillary bed at the terminus of each artery. When selecting among candidate flush solutions, the impact of this resistance on selected flow rate and pressure is an important consideration. If, for example, a saline flush were used, when collecting OCT imaging data, within one heart cycle the capillaries would fill with saline and the flow resistance would drop by a factor of 3 or more due to the viscosity change. To prevent blood from entering the imaging region, where the OCT data is collected, the local pressure has to equal or slightly exceed the native blood pressure. In this scenario, the saline flow rate would have to three or four times the native flow rate to maintain the condition of steady-state clearing.

A flow rate of this magnitude, (three or four times the native flow) is unacceptable for several reasons. For example, it can be dangerous for the patient because there is a risk of arterial wall rupture or trauma to weakened structures within the arterial wall such as thin-capped atheromas. Furthermore, an excessive flow rate will subject the catheter through which the flush is delivered to an equal and opposite force. This can cause unwanted catheter movement which, in turn, may result in arterial wall damage or unusable OCT scan data.

This effect was not previously recognized when using OCT to image coronary arteries. Saline or lactated Ringers solution (both having viscosities essentially equivalent to water) were used exclusively as a flush medium, and although extremely short imaging ‘windows’ were reported (about 1 or 2 heart cycles in length), the connection to capillary flow resistance was not established. Other approaches used a combination of occlusion (typically with a balloon as mentioned earlier), followed by saline flush. Since the vessel was occluded, low rates of saline injections could be used, however this came at the expense of a significantly more complex procedure, and the occlusion times typically lasted 30 seconds or more as the balloon inflation and deflation times were often longer than the actual imaging time. Such long occlusions carry risks of cardiac stress, usually clinically manifested as significant ECG changes. For this reason, occlusive imaging techniques have not found widespread adoption. Furthermore, the low saline flush rates produced pressures well below the native blood pressure distal to the balloon and hence arterial geometries (such as vessel size) in the imaged regions were no longer representative of the actual condition as a result of the pressure deviation from normal conditions.

For these reasons it is advantageous to non-occlusively flush and also flush with a solution that has comparable or larger viscosities than that of blood. Iodine-based radio-opaque dyes used in standard angiographic imaging have viscosities ranging from about 3 to about 5 cps and from 5 cps to about 10 cps (or more) at body temperature. As a result, these dyes represent a viable choice for a viscous flush. In addition, these solutions are already approved for coronary injections.

Dextran solutions, widely used as plasma expanders before major surgery (such as coronary bypass procedures) are also suitable flush solutions when collecting OCT scan data. This follows because dextran solutions have viscosities in the range of from about 3 cps to about 6 cps, depending on the dextrose levels. Dextrans do not suffer the potential renal complications associated with high levels of iodine, a well-known risk of the radiographic contrast agents. The contrast agent can be radio opaque. In some embodiments, the flush solution comprises a dextran concentration that ranges from about 5% to about 20%, with a molecular weight of between about 20,000 to about 100,000 Daltons. For example, in one embodiment, dextran has a molecular weight of about 40,000 Daltons and is in a 10% solution (about 10 g Dextran per about 100 ml of 5% dextrose hydrous solution). This formulation is in common use and termed ‘Dextran 40’. In another embodiment, dextran refers to a general class of water-soluble polymer of glucose of high molecular weight. Further, in another embodiment, as used herein dextran or dextrans refer to any of a group of long-chain glucose polymers with various molecular weights that are used in isotonic sodium chloride solution for the treatment of shock, in distilled water for the relief of the edema of nephrosis, and/or as plasma volume expanders in a solution of dextrose. In one embodiment, the contrast solution includes an iodine concentration from about 150 mg/ml to about 400 mg/ml

For purposes of OCT imaging, there exists a trade-off between the viscosity of the flushing fluid and the ability to thoroughly remove vestigial blood from the walls of the arteries and arterial structures such as branch points. Due to the Poisson distribution of blood velocities (highest in the center and near-zero at the lumen wall interface), effective clearing at the lumen-wall interface is not easy to obtain. Thus, a careful balance must be chosen between flow rate, total delivered volume (iodine loading issues), and overall clearing quality. For example, Visipaque 320™ (GE Healthcare), has the highest viscosity available in a common contrast agent, about 12 cps at body temperature and thus affords the lowest flow rate and smallest volumes required. However, Visipaque 320™ is ill-suited for removing blood cells near the lumen walls, stent struts, stenosis, and so forth. Saline delivers outstanding image quality at the expense of clinically unacceptable flush rates. Viscosities of up to about 8 or about 9 cps can be used, but image quality near the luminal boundary will diminish.

For practical reasons, it is worth noting that contrast agent viscosities have a strong temperature dependence, and can vary by a factor of 2 from room temperature to body temperature. Hence, the required input pressure to deliver room-temperature contrast solution can be quite high, limiting manually-achievable flow rates. In some applications this issues is resolved by maintaining the contrast agent at body temperature for this reason, as well as to avoid any effects of locally injecting ‘cold’ contrast into the coronaries. However, given the length of the guiding catheter, the injectate will be close to body temperature even if it started at room temperature. Visipaque 320™ has a room temperature viscosity of over 20, but drops to about 12 at body temperature, for example. In contrast, dextran solutions do not suffer from this strong temperature dependence. Unlike contrast solution, dextran offers several advantages. In one embodiment, a flush that includes dextran is systemically well-tolerated even in volumes measured in liters, and costs a small fraction of an iodine-based flush solution. Hence dextran represents a preferred candidate flush solution in one embodiment.

The flush catheter includes a catheter body, having a hollow inner cavity. The inner cavity is configured to communicate with a source of flush solution. The flush solution used may be, for example, sterile physiological saline, pure contrast solution, or a mixture of sterile saline and angiographic contrast solution. Other fluids may also be appropriate based on the particular application. One or more openings may be formed in an outer surface of the catheter body and may be arranged radially around a periphery of the catheter body in one or more rows. In one embodiment, the openings are arranged at an angle to facilitate flush direction.

The flush catheter further includes a sheath. According to one embodiment of the invention, the sheath can be formed of a thin piece of material of slightly larger inner diameter than an outer diameter of the catheter body. The sheath is positioned over the one or more openings and may be attached to the catheter body with an attaching means. In one embodiment, the sheath is attached to the catheter body at only one end, thus creating an annular volume open at the other end and extending along a length of the catheter body. In one embodiment, the sheath is attached to the catheter body at one end creating an annular volume open on an end facing a proximal end of the catheter when inserted into an artery, vessel, or other orifice, and extending along a length of the catheter body.

The flush catheter according to the invention may include a minirail delivery system at a distal end. In the case of a minirail delivery system, the one or more holes would be positioned a distance proximal to where the minirail attaches to the flush catheter.

For use with humans or animals, the sheath is preferably formed of a biocompatible material. For OCT or other imaging applications, the sheath is preferably transparent to allow light or other electromagnetic radiation to pass therethrough. In one embodiment, the sheath is formed of transparent polyethylene terephthalate (PET), although other materials may be appropriate based on the particular application.

Upon operation, the flush catheter is introduced into an artery, vessel, or other orifice of a patient. Flush solution provided by a flush solution source in communication with the inner cavity is directed through the inner cavity and radially outward through the one or more openings. The flush solution is then directed axially along an outer surface of the catheter body by the flow directing sheath.

That is, the flush solution introduced into the catheter from the proximal end flows radially out of the holes and is directed by the sheath along the outer surface of the flush catheter in a proximal direction. As the flush solution interacts with the blood flowing from the proximal end to the distal end of the flush catheter, it spreads out in the artery, vessel, or other bodily cavity or orifice, effectively substantially clearing a volume of the artery, vessel, or other bodily cavity or orifice.

For example, in the case of a blood vessel, by using an appropriate amount of flush solution an entire cylindrical volume may be substantially cleared of blood, using a flush flow rate significantly less than the flow rate of blood in the vessel. The ability to substantially clear a vessel of blood in an extended area while using a minimal rate of flush solution is one of the advantages of the invention.

The size and location of the one or more openings relative to an open end of the sheath may be chosen to allow for a substantially radially uniform flow of the flush solution from the sheath. By varying an inner diameter of the sheath and/or the catheter, a thickness of the annular gap may be modified, allowing for an average axial velocity of the flush solution to be controlled for a given flush flow rate. In one embodiment, the sheath is optional and enhanced flushing is achieved by using a viscous flush solution as outlined below.

By doing this, the momentum of the flush solution (proportional to Average Velocity×Mass Flow Rate of the flush solution) leaving the sheath may be varied to counteract the momentum of the blood flowing in the vessel. By raising the average velocity of the flush solution, a smaller mass flow rate can still counteract a larger but slower moving mass flow rate of blood. By varying the gap between the sheath and flush catheter, the momentum of the flush solution can be tuned to give the optimal length of cleared volume proximal to the sheath for a given application while using a minimal amount of flush solution.

Because all of the flush solution leaves the flush catheter in an axial direction, there is little worry of producing damage to the arterial or vessel wall. In addition, the proximity of the flush solution flow to the outer surface of the flush catheter substantially clears the outer surface of blood.

A specific embodiment of a flush catheter according to the invention will now be discussed in detail below. The following discussion teaches using the flush catheter in combination with an imaging catheter/probe, such as an image catheter/probe associated with an OCT device. However, the invention can be applied to other applications for which controlled flushing of an area is desirable.

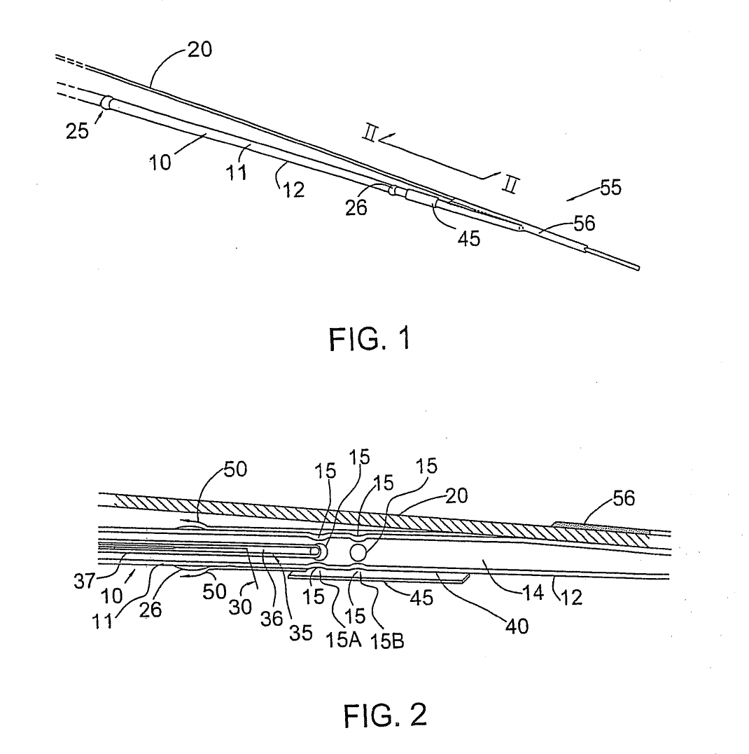

Further, the flush catheter of FIGS. 1-7 is shown used with a minirail delivery system. However, other delivery systems may also be appropriate.

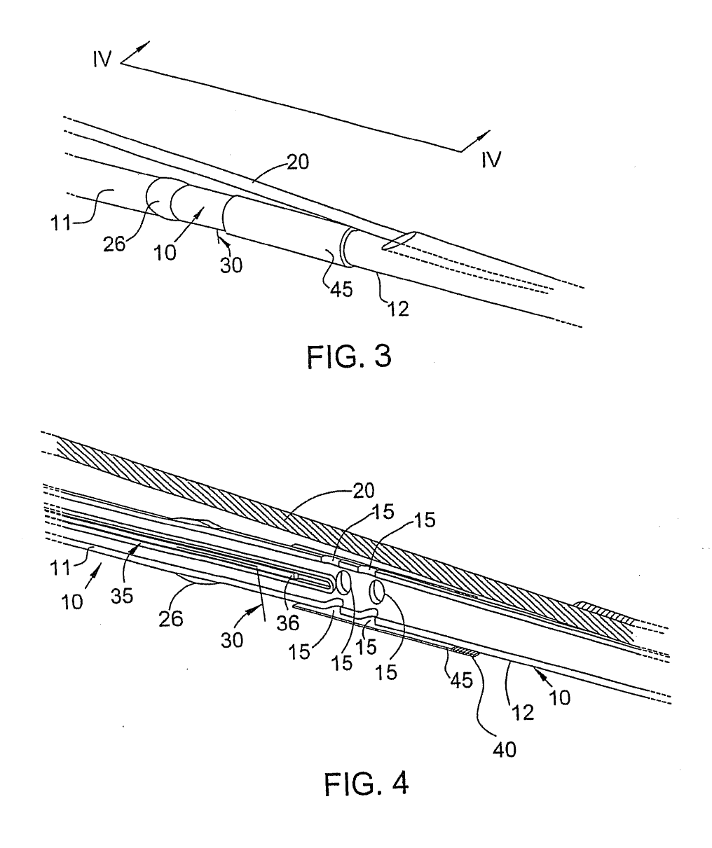

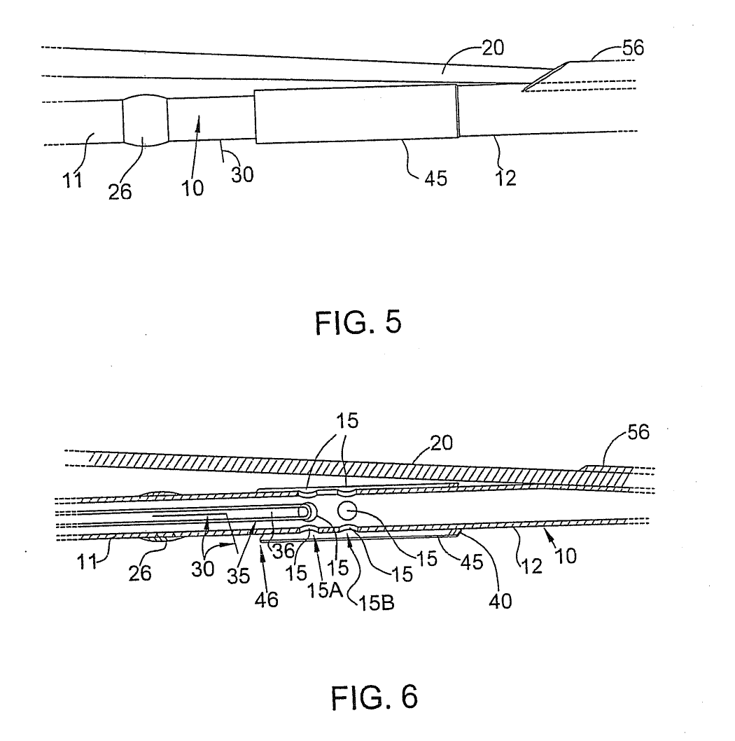

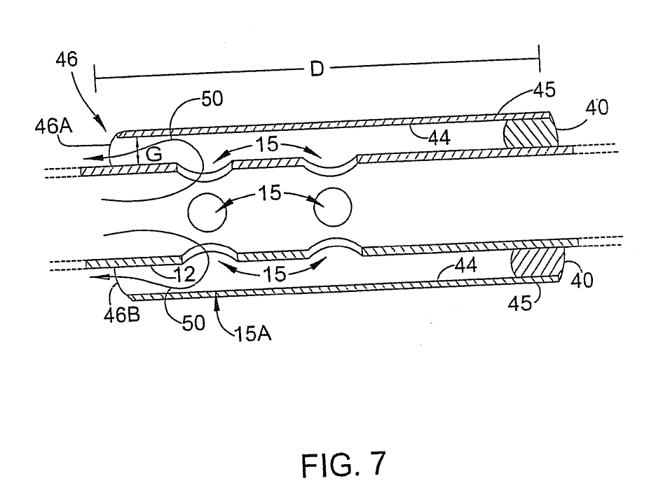

FIG. 1 is a schematic, partial, side, perspective view of a flush catheter implemented in combination with an imaging probe according to an embodiment of the invention. FIG. 2 is a schematic, partial, side, cross-sectional view of the flush catheter implemented in combination with an imaging probe of FIG. 1. FIG. 3 is another schematic, partial, side, perspective view of the flush catheter implemented in combination with an imaging probe of FIG. 1. FIG. 4 is another schematic, partial, side, cross-sectional perspective view of the flush catheter implemented in combination with an imaging probe of FIG. 1. FIG. 5 is a schematic, partial, side view of the flush catheter implemented in combination with an imaging probe of FIG. 1. FIG. 6 is a schematic, partial, side, cross-sectional view of the flush catheter implemented in combination with an imaging probe of FIG. 1. FIG. 7 is an enlarged, schematic, side, cross-sectional view of the sheath according to the invention.

FIG. 1 shows a flush catheter assembly 1 comprising a flush catheter 10. In the embodiment of FIG. 1, the flush catheter 10 is shown used in combination with a minirail delivery system 55. The minirail delivery system 55 includes a flexible tip 56 provided as part of the flush catheter 10 or configured to attach to the flush catheter 10, removably or permanently. The flexible tip 56 is configured to receive a guide wire 20, as shown in FIG. 1. The guide wire 20 allows the flush catheter 10 to be guided into an artery, vessel or other bodily cavity or orifice by a surgeon or other user.

For OCT imaging use, it is preferable that the flush catheter be made of a material that is transparent to the wavelengths of light used. For use with humans or animals, it is preferably that the flush catheter be made of a material that is biocompatible. One appropriate material that is both transparent and biocompatible is clear thermoplastic, one example of which is Polyester Block Amide, known as PEBA. However, other materials may also be appropriate.

The flush catheter 10 includes a catheter body 11 having an inner cavity 14. The inner cavity 14 is configured to communicate with a source of flush solution (not shown). The flush solution used may be, for example, sterile physiological saline, pure contrast solution, or a mixture of sterile saline and angiographic contrast solution. Other fluids may also be appropriate based on the particular application.

The inner cavity 14 is configured to receive an imaging core 35. The imaging core 35 includes an outer casing 37 in which an imaging probe 36, for example, a wire or optical fiber, is disposed. The imaging probe 36 is designed to output a beam of light 30 radially. The beam of light extends down a length of the imaging probe 36 and is deflected radially by a mirror 38. The imaging probe 36 may be rotated within the imaging core 35 to provide a disk-like scan of a target, such as an inner wall of an artery, vessel, or other bodily cavity or orifice. The imaging probe 36 may then be pulled lengthwise to scan a length of the target. That is, the imaging core 35 may be moved axially between a position underneath the flush sheath 45 proximal a distal marker band 26 to the proximal marker band 25. In this way, a survey may be made of a length of the wall of the artery, vessel, or other bodily cavity or orifice.

The imaging core 35 and imaging probe 36 are both preferably formed of a transparent material to allow the light beam 30 to pass therethrough. For example, the imaging core may be formed of polyester block amide, known as PEBA, onylon and the imaging probe may be formed of, for example, silica glass. However, other materials may also be appropriate.

As mentioned above, the flush catheter 10 further includes distal and proximal marker bands 25, 26, which may be raised as in the embodiment of FIG. 1. The marker bands 25, 26 are configured to allow a user to control the position of the flush catheter 10 and/or imaging probe 36. For example, the marker bands may be configured to be visible on, for example, an angiogram and may be used to find the position of the catheter in, for example, an arterial system. Also, if the marker bands are opaque to the O.C.T. probe, they provide a reference during pullbacks.

The flush catheter further includes one or more openings 15, as shown in FIG. 2, disposed in the catheter body 11. The one or more openings may be formed in an outer surface 12 of the catheter body 11 and may be arranged radially around a periphery of the catheter body 10 in one or more rows 15A, 15B.

Further, the flush catheter 10 further includes a sheath 45. The sheath 45 at least partially covers the one or more openings 15. The sheath 45 may comprise a thin piece of material and may be in the form of a cylinder disposed around the outer surface 12 of the catheter body 11 and extending a predetermined distance D along the length of the catheter body, as shown in FIG. 7.

The sheath 45 may be attached to an outer surface 12 of the flush catheter 10 by an attaching means 40, such as an adhesive. In one embodiment, the sheath is attached to the catheter body 11 at one end creating an annular volume open on an end facing a proximal end of the catheter 10 when inserted into an artery, vessel, or other bodily cavity or orifice. A gap G is formed between the inner surface 44 of the sheath 45 and the outer surface 12 of the catheter body 11.

In operation, the flush solution from a source (not shown) is pumped into and through inner cavity 14 and is expelled through opening(s) 15. The flush solution expelled through opening(s) 15 is directed by sheath 45 to flow along the outer surface 12 of the flush catheter 10, as shown by reference numeral 50 in FIGS. 2 and 7, forming a flush zone extending from the opening(s) 15 along the outer surface 12 of the flush catheter 12 to at least the distal marker band 25.

By varying a distance of the gap G formed between the sheath 45 and the opening(s) 15, the flow 50 can be controlled. That is, flush solution introduced into the flush catheter 10 from the proximal end flows radially out of the opening(s) 15 and is directed by the sheath 45 along the outer surface 12 of the flush catheter 10 in a proximal direction. The flush solution leaves the sheath 45 moving axially in a proximal direction. As the flush solution interacts with blood and/or other matter coming from the proximal to the distal end it will begin to spread out in the artery, vessel, or other bodily cavity or orifice, effectively substantially clearing a volume of the artery, vessel, or other bodily cavity or orifice of blood and/or other matter. The distal and/or proximal marker bands 25, 26 may be contoured to avoid blocking the flow of the flush solution along the outer surface 12 of the flush catheter 10. In addition, the distal and/or proximal marker bands 25, 26 may be sized to effectively prevent open edges 46A, 46B of the sheath 45 from contacting the walls of the artery, vessel, or other bodily cavity or orifice, minimizing the chances of damage when moving the entire flush catheter in a proximal direction.

By using an appropriate amount of flush solution, an entire cylindrical volume between the two marker bands 25, 26 may be substantially cleared of blood and/or other matter creating a flush zone, using a flush flow rate significantly less than the flow rate of blood in the artery, vessel, or other bodily cavity or orifice. The ability to substantially clear an artery, vessel, or other bodily cavity or orifice of blood and/or other matter in an extended area while using a minimal rate of flush solution is one of the advantages of the invention.

The size and location of the opening(s) 15 relative to the open end 46 of the sheath 45 may be chosen to allow for a substantially radially uniform flow of the flush solution from the sheath 45. By varying an inner diameter of the sheath 45 and/or the flush catheter 10, a thickness of the annular gap G may be modified, allowing for an average axial velocity of the flush solution to be controlled for a given flush flow rate. By doing this, the momentum of the flush solution (proportional to Average Velocity×Mass Flow Rate of the flush solution) leaving the sheath 45 may be varied to counteract the momentum of the blood and/or other matter flowing in the artery, vessel, or other orifice. By raising the average velocity of the flush solution, a smaller mass flow rate can still counteract a larger but slower moving mass flow rate of blood and/or other matter. By varying the gap G between the sheath 45 and flush catheter 10, the momentum of the flush solution can be tuned to give the optimal length of cleared volume proximal to the sheath 45 for a given application while using a minimal amount of flush solution.

Because all of the flush solution leaves the flush catheter 10 in an axial direction, there is little worry of producing damage to the walls of the artery, vessel, or other bodily cavity or orifice. In addition, the proximity of the flush solution flow to the outer surface 12 of the catheter 10 substantially clears the outer surface 12 of blood and/or other matter, resulting in a substantially clear image produced by the imaging probe 36.

Although the details of the flush catheter according to the invention have been optimized for its use in an OCT application, it is obvious that it may be easily modified for use in other applications, in particular where a complete flush is desired while using a minimum amount of flush solution.

Further, the design allows the flush zone to be placed anywhere along the flush catheter, merely by moving the positions of the opening(s) 15 and sheath 45. In applications where the flush catheter is introduced in the opposite direction, i.e. blood flow is toward a distal end of the flush catheter, the sheath may be reversed to provide effective flushing.

Further, by varying the gap between the sheath and the flush catheter, the average velocity of the flush solution leaving axially from the sheath may be controlled for a given flush rate. Additionally, by varying the number, size, and location of the opening(s) relative to the open end of the sheath, substantially non-uniform flows may be achieved for special applications.

The foregoing embodiments and advantages are merely exemplary and are not to be construed as limiting the invention. The present teaching can be readily applied to other types of apparatuses. The description of the invention is intended to be illustrative, and not to limit the scope of the claims. Many alternatives, modifications, and variations will be apparent to those skilled in the art. In the claims, means-plus-function clauses are intended to cover the structures described herein as performing the recited function and not only structural equivalents but also equivalent structures.

What is claimed is:

1. A flush catheter configured to be introduced into a lumen to create an optically transparent flush zone, comprising:
a catheter body configured to be introduced into a lumen and an inner cavity, the inner cavity being configured to communicate with a proximal source of flush solution and expel the flush solution at a distal end of the catheter;
an image probe assembly contained within the catheter body; and
a plurality of openings provided in the catheter body configured to expel therethrough the flush solution; wherein when flush solution is expelled through the plurality of openings into the lumen, the flush solution is directed to flow toward a proximal end of the catheter body, and wherein a flush solution flow rate is configured such that a volume flow rate of the expelled flush solution is substantially equivalent but opposite to that of locally flowing blood thereby creating the optically transparent flush zone along a length of the lumen such that non-occlusive optical imaging of the lumen can be performed by the image probe assembly.

2. The flush catheter of claim 1 wherein the flush solution comprises iodine having a concentration that ranges from about 150 mg/ml to 400 mg/ml.

3. The flush catheter of claim 1, wherein the flush solution has a viscosity that ranges from about 3 cps to about 9 cps at body temperature.

4. The flush catheter of claim 1, wherein a viscosity of the flush solution does not vary substantially with temperature.

5. The flush catheter of claim 1, wherein the flush solution comprises dextran.

6. The flush catheter of claim 1, wherein the flush solution comprises a dextran concentration that ranges from 5% to 20%, with a molecular weight of 20,000 to 100,000 Daltons.

7. A method of flushing a lumen of interest having a first diameter and a lumen wall; the method comprising the steps of:
selecting a flush solution such that the flush solution lowers a fluid removal rate of a plurality of terminating lumens, the terminating lumens branching from and in fluid communication with the lumen of interest, at least one of the terminating lumens having a second diameter, the second diameter smaller than the first diameter;
flushing the lumen with the flush solution; and
collecting optical tomography scan data relative to a portion of the lumen wall.

8. The method of claim 7 wherein the flush solution has been further selected to remove blood near the portion of the lumen wall.

9. The method of claim 7 wherein the flush solution comprises dextran.

10. The method of claim 7 wherein the terminating lumens are capillaries.

11. The method of claim 7 wherein the flush solution has a viscosity that ranges from about 3 cps to about 9 cps at body temperature.

12. The method of claim 7 wherein the flush solution is a radio-opaque contrast solution.

13. The method of claim 12 wherein an iodine concentration of the contrast solution ranges from about 150 mg/ml to 400 mg/ml.

14. The method of claim 12 wherein the contrast solution comprises iodine having a concentration from about 150 mg/ml to about 400 mg/ml.
